(19) United States
(12) Patent Application Publication
(10) Pub. No.: US 2024/0032875 A1
(43) Pub. Date: Feb. 1, 2024

(54) Composite Phonocardiogram Visualization on an Electronic Stethoscope Display

(71) Applicant: 3M INNOVATIVE PROPERTIES COMPANY, St. Paul, MN (US)

(72) Inventor: William Bedingham, Woodbury, MN (US)

(21) Appl. No.: 18/487,190

(22) Filed: Oct. 16, 2023

Related U.S. Application Data
(63) Continuation of application No. 17/387,413, filed on Jul. 28, 2021.
(60) Provisional application No. 63/059,265, filed on Jul. 31, 2020.

(51) Int. Cl. A61B 5/00; A61B 7/04

(52) U.S. Cl. CPC A61B 5/742; A61B 7/04; A61B 2562/0204

(57) ABSTRACT

Aspects of the present disclosure relate to a method of displaying a portion of an acoustic signal on an electronic stethoscope. The method can include receiving the acoustic signal from an acoustic sensor of the electronic stethoscope and an electrical signal from a sensing module of the electronic stethoscope. The method can include identifying a trigger point associated with the electrical signal. The method can include generating acoustic information from the acoustic signal and synchronizing the acoustic information to the trigger point for the cardiac cycle. The method can include generating a phonocardiogram as a composite phonocardiogram based on the synchronized acoustic information. The method can include displaying a representative portion of the composite phonocardiogram in real-time for a duration of a first period of time on a display that is integral to a chest piece of the electronic stethoscope.

### BACKGROUND

A variety of devices have been developed to detect sounds produced by the body, such as heart and lung sounds. These devices range from primarily mechanical devices, such as a stethoscope, to various electronic devices, such as microphones and transducers. The stethoscope, for example, is a fundamental tool used in the diagnosis of diseases and conditions of the cardiovascular system. It serves as the most commonly employed technique for diagnosis of such diseases and conditions in primary health care and in circumstances where sophisticated medical equipment is not available, such as remote areas.

Clinicians readily appreciate that detecting relevant cardiac symptoms and forming a diagnosis based on sounds heard through the stethoscope, for example, is a skill that can take years to acquire and refine. Heart sounds are often separated from one another by relatively short periods of time, and abnormal sounds that may be characteristic of cardiac disorders may be less audible than normal heart sounds. In some examples, a system may be used to generate a graphical representation (e.g., a phonocardiogram) of detected heart sounds.

### BRIEF SUMMARY

Aspects of the present disclosure can relate to a method of displaying a portion of an acoustic signal on an electronic stethoscope. The method can include receiving the acoustic signal from an acoustic sensor of the electronic stethoscope and an electrical signal from a sensing module of the electronic stethoscope. The method can also include identifying a trigger point associated with the electrical signal (using the processor of the electronic stethoscope). The electrical signal can be derived from a cardiac cycle from a plurality of cardiac cycles, the plurality of cardiac cycles is associated with a (individual) patient. The method can also include generating acoustic information from the acoustic signal and synchronizing the acoustic information to the trigger point for the cardiac cycle. The method can also include generating a phonocardiogram as a composite phonocardiogram based on the synchronized acoustic information. The composite phonocardiogram can be generated for the cardiac cycle. The method can also include displaying a representative portion of the composite phonocardiogram on a display of the electronic stethoscope in real-time for a duration of a first period of time. The display is integral to a chest piece or a housing of a chest piece of the electronic stethoscope and the chest piece is configured for handheld manipulation by a clinician.

Additional aspects of the present disclosure relate to an electronic stethoscope and systems thereof. The electronic stethoscope can include chest piece formed from a housing. The electronic stethoscope can include a display integral with the housing of the chest piece. The chest piece can also include a sensing module, an acoustic sensor, and a processor communicatively coupled to the display, the sensing module, a memory and the acoustic sensor. The memory can store instructions that, when executed by the processor, configure the electronic stethoscope to receive an acoustic signal from the acoustic sensor of an electronic stethoscope and an electrical signal from the sensing module of the electronic stethoscope, identify a trigger point associated with the electrical signal that is derived from a cardiac cycle from a plurality of cardiac cycles(the cardiac cycle is associated with a patient), generate acoustic information from the acoustic signal, synchronize the acoustic information to the trigger point for the cardiac cycle, generate a phonocardiogram as a composite phonocardiogram based on the synchronized acoustic information, and display a representative portion of the composite phonocardiogram on the display in real-time for a duration of a first period of time.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A illustrates an electronic stethoscope 100 in accordance with one embodiment.

FIG. 1B illustrates an aspect of the electronic stethoscope 100 in accordance with one embodiment.

FIG. 1C illustrates an aspect of the electronic stethoscope 100 in accordance with one embodiment.

FIG. 2 illustrates a method 200 in accordance with one embodiment.

FIG. 3 illustrates a subroutine block 300 in accordance with one embodiment.

FIG. 4 illustrates an exemplary flowchart in accordance with one embodiment.

FIG. 5 illustrates an exemplary flowchart in accordance with one embodiment.

FIG. 6 illustrates exemplary composite phonocardiograms in accordance with one embodiment.

FIG. 7 illustrates a system 700 in accordance with one embodiment.

FIG. 8 illustrates an embodiment of an electronic stethoscope 800 in accordance with one embodiment.

### DETAILED DESCRIPTION

Aspects of the present disclosure relate to displaying a cardiac cycle of a patient directly on an electronic stethoscope in real-time. The cardiac cycle can be formatted to be displayed on a smaller displayable area found on an electronic stethoscope. For example, the display can have a displayable area of less than 1000 square millimeters.

While some aspects may be described in U.S. Pat. No. 9,521,956, issued Dec. 20, 2016, which is incorporated by reference, various aspects of the present disclosure relate to a display that can be integral with a chest piece of an electronic stethoscope and capable of being held by a clinician while operating the electronic stethoscope. However, one issue with this approach is that the cardiac cycle may or may not be synchronized. For example, the phonocardiogram can be displayed at any phase of the heart cycle and/or the phonocardiogram on the display may be continuously moving which can make it hard for a clinician to identify cardiac acoustic features. In at least one embodiment, a representative portion can be specifically formatted to be viewed on the display by the clinician so that the clinician can evaluate the cardiac cycle. For example, as opposed to viewing a plurality of cardiac cycles on the integral display, the clinician can view a single cardiac cycle (or portions thereof) expanded to the displayable area.

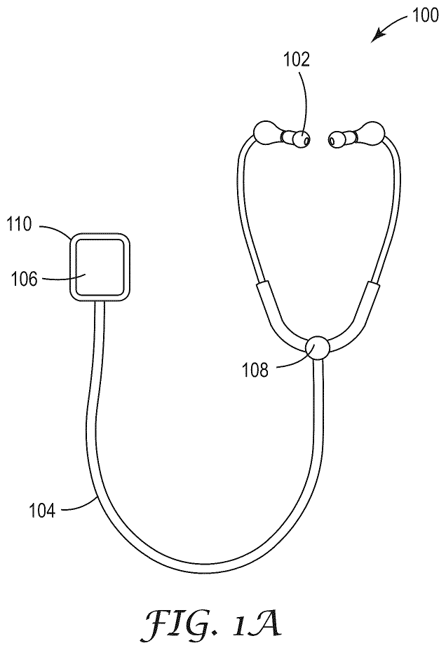

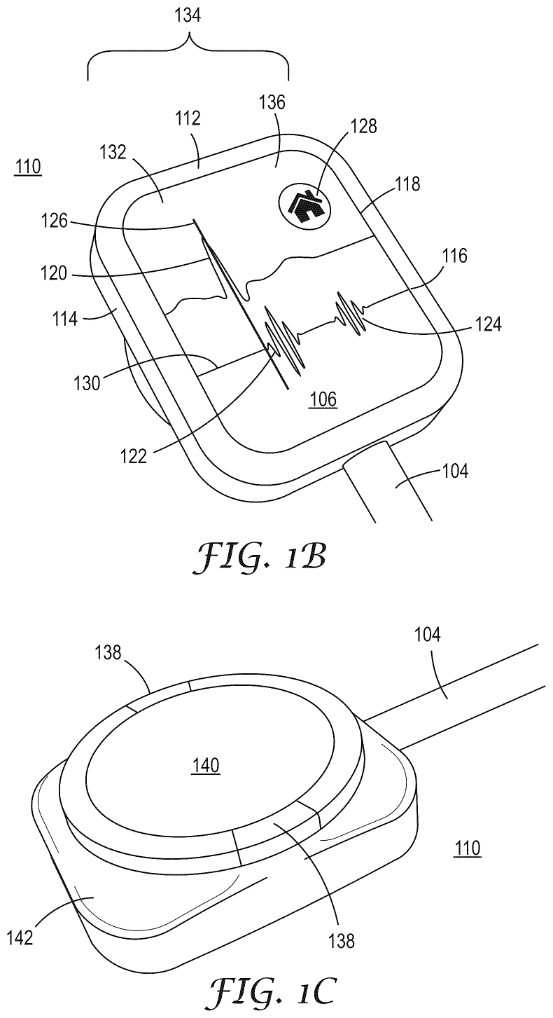

FIG. 1A-FIG. 1C illustrate an electronic stethoscope 100. Examples of some components described herein can be found in electronic stethoscopes from 3M (Maplewood, MN).

The electronic stethoscope 100 can be modified to include a display 106 capable of displaying a composite phonocardiogram. For example, a resolution of the display 106 can be sufficient for a human user to read a composite phonocardiogram 132 at a distance of less than 3 feet. In at least one embodiment, the display 106 can have a resolution of at least 80 pixels per square inch, or at least 150 pixels per square inch. In at least one embodiment, the display 106 can have a resolution of at least 100 dots per inch (dpi) or at least 200 dpi. The display 106 can be configured to be manipulatable by a clinician with one hand. In at least one embodiment, the display 106 can have a small displayable area. For example, the displayable area can be no greater than 1000 square millimeters.

FIG. 1A shows the electronic stethoscope 100 as a whole. The electronic stethoscope 100 can have a chest piece 110 which has a housing 114 configured to contain the electrical components of the electronic stethoscope 100. For example, the housing 114 can be configured to contain the processor, memory, display 106, acoustic sensor 140, and sensing module 138 described herein. The housing 114 can secure the display 106 such that the display 106 does not remove from the chest piece 110.

To provide sound of a patient's heartbeat or any auditory alarms, the electronic stethoscope 100 can transmit sound via a sound transmission device 104. The sound transmission device 104 can be tubing coupled to a speaker in the housing 114 . For example, the tubing can be pneumatically and acoustically coupled to the eartips 102 which may be conformable to the ear canal of the clinician. The eartips 102 can join together at the yoke 108 to resemble a traditional stethoscope. In at least one embodiment, sound transmission device 104 can transmit the sound as an electrical signal that is used to drive speakers located at the eartips 102. For example, the sound transmission device 104 can be electrical wiring leading to speakers proximate to the eartips 102. In this example, the eartips 102 can be electronic earbuds. In at least one embodiment, the sound transmission device 104 can be a locally wireless communication between the chest piece 110 and the eartips 102.

FIG. 1B illustrates a more expanded view of the chest piece 110. The chest piece 110 illustrates a clinician side 112 which faces in a direction opposite to the patient. The clinician side 112 can be generally opposite of the sensor placement of the acoustic sensor 140.

The housing 114 can have a display 106 contained therein. The display 106 can be configured to project a user interface 136 for presenting information to the user, such as a visual (or audible) representation of the composite phonocardiogram 132. The user interface 136 can have a navigation menu 128 to navigate through different functions on the electronic stethoscope 100. The user interface 136 can display both normal and pathological feature identification, numerical metrics (e.g., heart rate, heart period, or breaths per minute, etc.), and text ID (e.g., S1, S2, S3, S4, murmur, click, etc.). In at least one embodiment, a single cardiac cycle without feature identification or numerical metrics is shown.

The user interface 136 may include a display element and/or a speaker for sound interaction or a haptic transducer to provide vibratory touch information of feedback. The user interface 136 may include one or more input devices and/or output devices so that the user can communicate with electronic stethoscope 100. In one example, the user interface 136 can include a remote control from another device which may provide remote control and remote listening to reduce the transmission of microorganisms. In one example, the user interface 136 may be a touch screen interface. In other examples, the user interface 136 may include a display and one or more buttons, pads, joysticks, mice, tactile device, or any other device capable of turning user actions into electrical signals that control electronic stethoscope 100. In any example, the clinician may interact with the user interface 136 to provide input prior to or during the processes described herein. In some examples, electronic stethoscope 100 may at least partially command acoustic sensor or the sensing module 138.

The composite phonocardiogram 132 can include acoustic signal 116 and electrical signal 118 (e.g., electrocardiogram) which both can correspond to a single cardiac cycle. An aspect of the present disclosure is that a representative cardiac cycle is displayed over a period of time 134 on a majority of the displayable area of the display. This can allow a clinician to quickly view a representative cardiac cycle without having to view a series of cardiac cycles. This is particularly beneficial on displays with a smaller displayable area.

The acoustic signal 116 may be a raw acoustic signal as detected by an acoustic sensor. In other words, acoustic signal 116 may include noise from breathing sounds, gastrointestinal sounds, movement of the acoustic sensor on the skin, and any other unintended sounds. Acoustic signal 116 may still include identifiable features such as the S1 heart sound (acoustic feature 122) and features as the S2 heart sound (acoustic feature 124). In at least one embodiment, the acoustic signal can be processed or enhanced before displaying the data (e.g., by digital filtering, amplification, integration/differentiation, rectification, power envelope, etc.).

The period of time 134 can commence at time start 130 which can be triggered by an electrical signal 118. The electrical signal 118 may provide a representation of the electrical activity of the heart. The electrical signal 118 can include a trigger point 120 which can set the time start 130. The acoustic signal 116 can be synchronized off of the trigger point 120 as described herein.

The composite phonocardiogram 132 can indicate when one or more physiologically significant features of the acoustic signal 116 are present. For example, electronic stethoscope 100 may analyze the acoustic signal 116 for S1 and/or S2 heart sounds. Part of the display 106 may blink each time the S1 heart sound is present. Alternatively, part of the display 106 may light up with different colors to differentiate between S1 and S2 sound present in the acoustic signal 116. In another example, the display 106 can display different colors for different parameters, features, alerts, etc . . . In at least one embodiment, an auditory cue can be emitted at the trigger point 120, or some other predetermined feature. For example, at the trigger point 120, a sound can be emitted to indicate the time start.

In at least one embodiment, a cursor bar 126 can be displayed to mark the position of current time on the composite phonocardiogram 132 in real-time. For example, the chest piece 110 receives a first cardiac cycle and the cursor bar 126 moves from time start 130 to the right. As the cursor bar 126 moves, the portion to the left of the cursor bar 126 is displayed, but the portion to the right of the cursor bar 126 can be blank. In another example, the previously recorded cardiac cycle can be erased as the cursor bar sweeps to the right. Once the cursor bar 126 reaches the end of the first cardiac cycle, then the display 106 can reset such that the process can begin again with a second cardiac cycle. In at least one embodiment, the last cardiac cycle can be frozen, stored, processed further, or updated as the next cardiac cycle is recorded.

FIG. 1C illustrates another view of the chest piece 110 from the patient-facing side 142. The patient-facing side 142 may be the surface intended to contact the skin of a patient. Patient-facing side 142 may refer a portion of the housing 114.

The chest piece 110 can include an acoustic sensor 140 and sensing module 138 on the patient-facing side 142. The sensing module 138 and acoustic sensor 140 can acoustically/electrically couple to the sound transmission device 104.

The sensing module 138 can have two or more electrodes that detect electrical signals of the heart. In one example, the electronic stethoscope may be coupled to each of the electrodes via a respective wire. In another example, the electronic stethoscope may carry each of the electrodes on the housing 114 of the chest piece 110. For example, the electrodes can be metal half-rings having a gap as the insulator which can produce a differential voltage/current measurement. The electrodes can be protruding from a planar surface established by the acoustic sensor 140. The electrodes may be arranged to increase the distance between each electrode to improve the sensing vector between each electrode. Although two electrodes are shown, three or more electrodes may be provided.

The acoustic sensor 140 may detect acoustic signals from a heart of the patient. Acoustic sensor 140 may be disposed generally in the center of housing 114. In other examples, acoustic sensor 140 may be located away from the center of the housing 114.

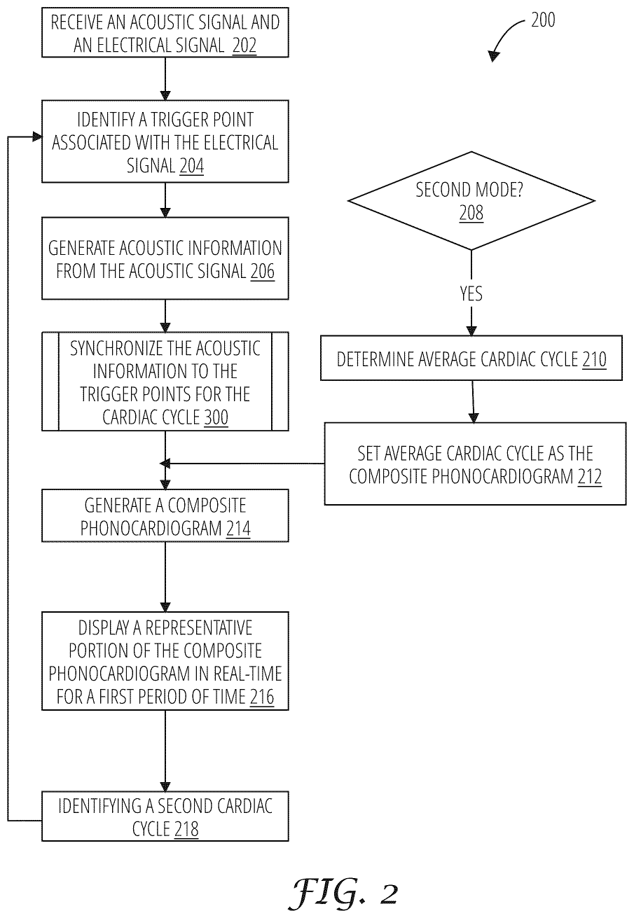

FIG. 2 illustrates a flowchart of a method 200 of the operations of the electronic stethoscope.

In block 202, the electronic stethoscope receives an acoustic signal from an acoustic sensor and an electrical signal from a sensing module. The acoustic signals and electrical signals can be received into the memory of the electronic stethoscope (e.g., recorded, and/or via Direct Memory Access) for later processing. In at least one embodiment, the receiving of the acoustic signal and the electrical signal can refer to either individual electrical readings corresponding to heart electrical readings, or acoustic readings, or collectively to a session involving a plurality of individual electrical or acoustic readings.

In block 204, the electronic stethoscope can identify a trigger point associated with the electrical signal. The electrical signal is derived from a cardiac cycle from a plurality of cardiac cycles. For example, the electronic stethoscope may detect an r-wave and use the r-wave detection as a trigger. In at least one embodiment, the trigger point can be in real-time to synchronize the new data acquisition. The samples of sound and electrical data points can be displayed in real-time or placed in an array for processing (e.g., averaging). In at least one embodiment, any of the electrical features can be used as a trigger point (e.g., q-wave or s-wave). Once the trigger point is identified, then the method 200 can continue on a rolling basis (e.g., continue evaluating the received electrical signal for additional trigger points). In one example, an embedded Pan-Tompkins algorithm can be used to identify the trigger point. In at least one embodiment, a plurality of trigger points can be determined.

In block 206, the electronic stethoscope can generate acoustic information from the acoustic signal. For example, the acoustic signal can be used to assemble a phonocardiogram.

In subroutine block 300, the electronic stethoscope can synchronize the acoustic information to the trigger point(s) for the cardiac cycle. The synchronization can be performed in real-time. For example, once the trigger point is identified, then anything after the trigger point can be associated with a first cardiac cycle. Once a second trigger point is identified, then anything after the second trigger point can be associated with a second cardiac cycle. In at least one embodiment, subroutine block 300 can represent a circular buffer where the memory is continuously being updated and the oldest data overwritten. For example, a valid trigger event occurs (e.g., the trigger point in the first cardiac cycle), then a percentage of the stored phonocardiogram or electrocardiogram data before the trigger is displayed (e.g., 20% of the total cardiac cycle). When available, the new data continues to be displayed and updated in the buffer. When a new trigger event occurs, then this process repeats at the new location in the circular buffer.

In block 214, the electronic stethoscope can generate a phonocardiogram as a composite phonocardiogram based on the synchronized acoustic information. For example, the composite phonocardiogram is generated for the cardiac cycle. The composite phonocardiogram can include both the acoustic signals and the electrical signals that are synchronized with each other. In at least one embodiment, the acoustic signals are automatically associated with a cardiac cycle based on the presence of the trigger point.

The detected electrical signals (e.g., an R-wave and/or T-wave of an electrocardiogram) may be used to synchronize acoustic signals (e.g., S1 and S2 heart sounds) from multiple cardiac cycles to reinforce the heart sounds. For example, the system may detect an r-wave and use the r-wave detection as a trigger for synchronizing the acoustic signals from each cardiac cycle. To reinforce the heart sounds, acoustic signals from each of multiple cardiac cycles may be averaged or summed to produce a composite phonocardiogram (PCG).

The composite phonocardiogram may be presented to a clinician by generating an audible signal of the composite phonocardiogram and/or generating a visual display of the composite phonocardiogram. For example, the electronic stethoscope may transmit detected electrical signals and acoustic signals to a computing device (e.g., a notebook computer, a tablet computer, or a mobile phone) for processing and generating the composite phonocardiogram.

A user interface of the electronic stethoscope may then present a graph of the composite phonocardiogram. In some examples, the user interface may color code or otherwise identify abnormal sounds and/or normal sounds within the composite phonocardiogram. In other examples, the user interface may include a speaker that generates an audible representation of the composite phonocardiogram. In this manner, the audible representation of the composite phonocardiogram may be played to the user alone or on top of (or overlaid with) real-time acoustic signals presented audibly to the user. In other examples, the electronic stethoscope may send an audio signal to a wireless headset, a wired headset, or via sound transmission device 104. The clinician may then listen to the audio signal and/or composite phonocardiogram using one of these devices. The audible and/or visual composite phonocardiogram may enable a clinician to identify heart sound pathologies, diagnose a patient, and/or learn how to identify different heart sound pathologies. In some examples, the composite phonocardiogram may expose audible events otherwise covered or masked by noise or other heart sounds.

In at least one embodiment, an average cardiac cycle can be set as the composite phonocardiogram in block 212.

For example, if a second mode is selected in decision block 208 by the clinician, then the electronic stethoscope can determine an average cardiac cycle in block 210. The average cardiac cycle may be a composite of a plurality of cardiac cycles. For example, the average cardiac cycle may not correspond to an exact cardiac cycle but rather an average of a plurality of cardiac cycles. In one embodiment, the average cardiac cycle is based on a rolling average of the plurality of previously recorded cardiac cycles. For example, the rolling average can be the 10 previously recorded cardiac cycles, the 20 previously recorded cardiac cycles, the 30 previously recorded cardiac cycles, or the 60 previously recorded cardiac cycles. The average can be based on the intensity of the electrical reading for both the acoustic features and electrical features at the same time or occurrence in the composite phonocardiogram. This averaging may reduce the presence of noise in the composite phonocardiogram that may otherwise be present in the acoustic signals for individual cardiac cycles. The composite phonocardiogram may represent the heart sounds during one cardiac cycle of the patient.

In block 216, the electronic stethoscope can display a representative portion of the composite phonocardiogram in real-time for a first period of time. In at least one embodiment, the electronic stethoscope reads the acoustic signal from the memory at the time start.

In at least one embodiment, the first period of time is related to a duration of a previously recorded cardiac cycle. For example, if the previously recorded cardiac cycle is 1 second, then the currently displayed cardiac cycle can also be 1 second. The previously recorded cardiac cycle in this example immediately precedes the currently displayed cardiac cycle. In another example, the previously recorded cardiac cycle can refer to any cardiac cycle that precedes the currently displayed cardiac cycle. For example, the previously recorded cardiac cycle can be the 20th cardiac cycle that preceded the currently displayed cardiac cycle. In at least one embodiment, the duration of the previously recorded cardiac cycle can be an average duration of a plurality of previously recorded cardiac cycles. In another example, the first period of time is related to a pulse rate of the patient, e.g., if the heart rate of the patient is 60 beats per minute, then the first period of time can be set to 1 second.

In at least one embodiment, the representative portion can be a segment of the composite phonocardiogram. For example, the representative portion can represent a single cardiac cycle or an average cardiac cycle (e.g., of a plurality of cardiac cycles). In one example, if a single cardiac cycle is displayed, then no other cardiac cycles besides the first cardiac cycle are displayed at the same time. If the representative portion is an average cardiac cycle, then the average cardiac cycle can be displayed for a second period of time. The second period of time can be predetermined and unrelated to the first period of time. For example, the second period of time can be set at least 5 seconds. In one example, the display can be adjusted to show just one cardiac cycle with a percentage of overlap from the previous and the upcoming cardiac cycle. The heart rate and period of the trigger interval can be constantly updated, and the display adjusted to map the current cardiac cycle.

The representative portion can display both the electrocardiogram and phonocardiogram that is synchronized off of the trigger point. The representative portion can also include a time period before the trigger point so that a QRS waveform of an electrical signal can be shown. In some embodiments, the time period can be set to a default time period before the trigger point (e.g., set to at no greater than 10 milliseconds before the trigger point or a percentage of the cardiac cycle).

In at least one embodiment, the representative portion can begin at the time start and end after the first period of time has elapsed. The representative portion can also be related to a cursor bar. For example, a cursor bar may scroll across the display as a function of time. The representative portion may be displayed based on the position of the cursor bar projected on the display. In at least one embodiment, displaying the cursor bar across the composite phonocardiogram can occur as a function of time. Thus, the cursor bar can be based on the time start. For example, at the time start, the cursor bar can be positioned at the time start and the representative portion can be blank. As the cursor bar moves from left to right, the representative portion can encompass an increasing amount of the cardiac cycle.

In block 218, the electronic stethoscope can identify a second cardiac cycle of the patient after the first period of time has elapsed. The second cardiac cycle can be identified based on the presence of a second trigger point that is determined similarly to the first trigger point. As a result, the electronic stethoscope can display the second cardiac cycle. In at least one embodiment, the second cardiac cycle can be displayed for a duration equivalent to the first period of time. The duration can be configured such that the display in block 216 is continuously flashing a new cardiac cycle. For example, a new image of a cardiac cycle can be displayed for every duration of a cardiac cycle (e.g., every 1 second). The flashing can be configured to allow the clinician to be able to discern the acoustic features. In at least one embodiment, the display can continuously scroll the composite phonocardiogram so that the image displayed has an animated composite phonocardiogram that has amplitudes that vary with respect to a time indication.

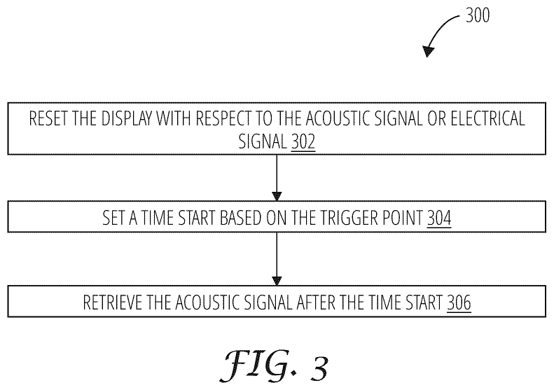

FIG. 3 illustrates an embodiment of subroutine block 300. Subroutine block 300 can relate to the synchronization of the electrical signal with the acoustic signal. In some aspects, block 302, block 304 and block 306 can be in any order.

In block 302, electronic stethoscope can reset the display with respect to the acoustic signal or electrical signal. For example, after the first period of time has elapsed, the electronic stethoscope can cause the display to become blank.

In block 304, the electronic stethoscope can set a time start based on the trigger point. For example, the R-wave can establish a trigger point and the electronic stethoscope can set the time start based on the R-wave. In block 306, the electronic stethoscope can retrieve the acoustic signal (from memory) after the time start. The electronic stethoscope can further wait for the first period of time to elapse or wait until the next trigger point.

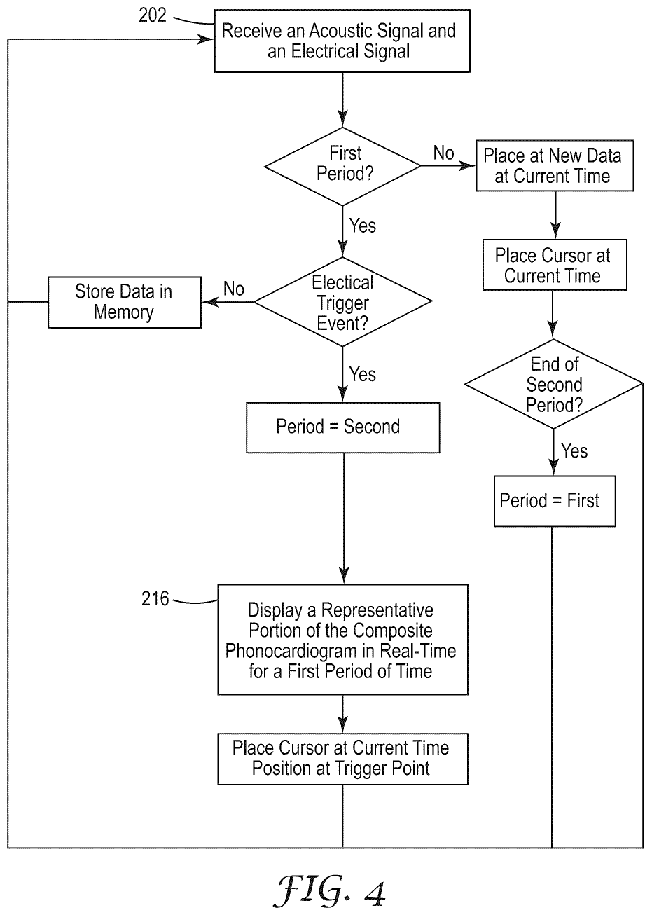

FIG. 4 illustrates another embodiment of method 200 representing a single-trace mode.

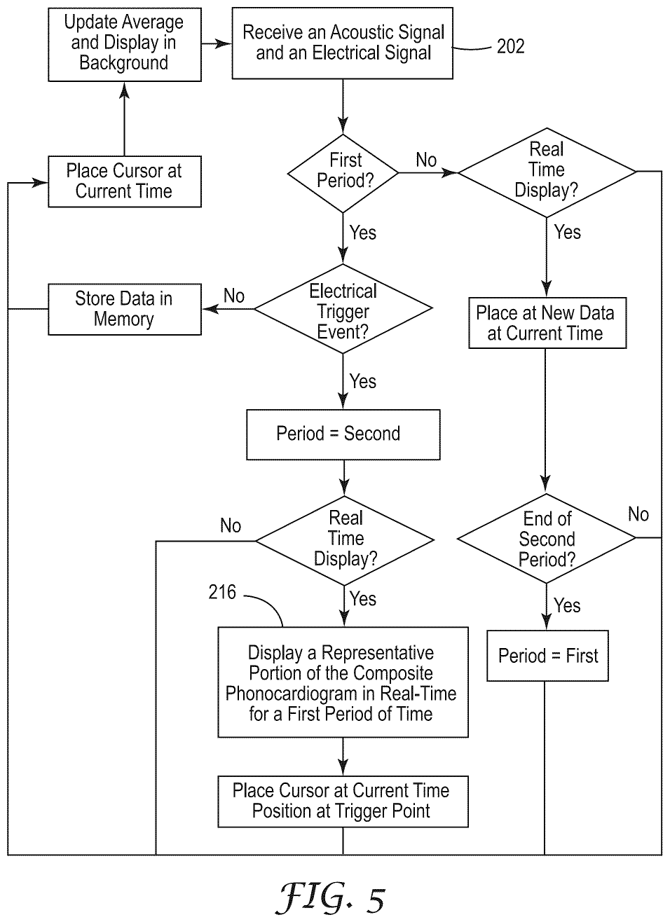

FIG. 5 illustrates another embodiment of method 200 representing an averaging (i.e., second mode).

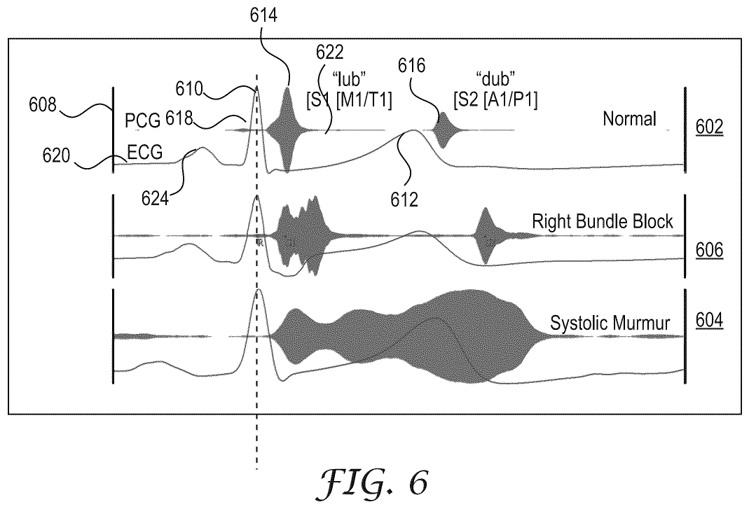

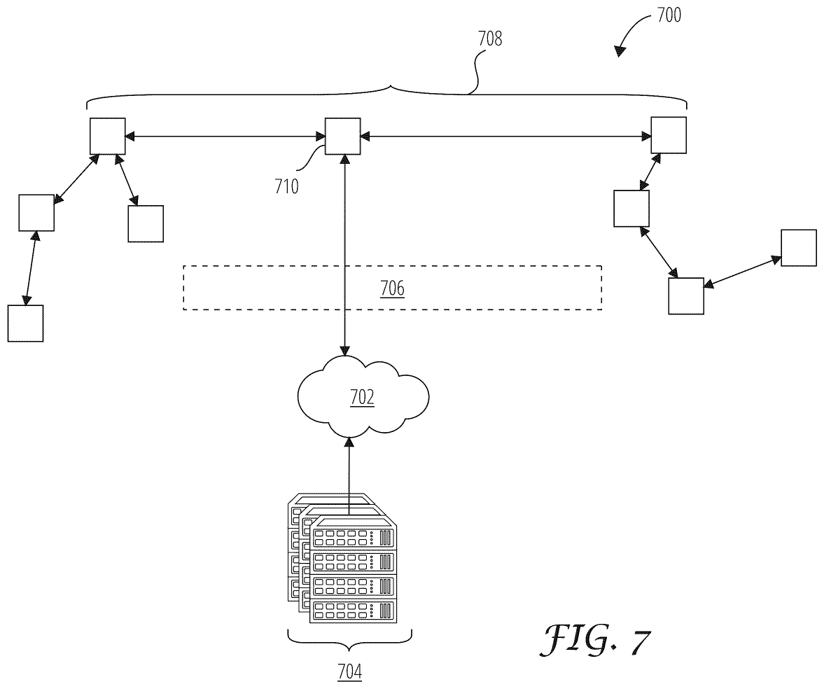

FIG. 6 illustrates sample composite phonocardiograms, composite phonocardiogram 602, composite phonocardiogram 604, and composite phonocardiogram 606. Composite phonocardiogram 602 can indicate a normal composite phonocardiogram, composite phonocardiogram 604 can represent a heart condition of right bundle blockage, and composite phonocardiogram 606 can represent a systolic murmur. Each composite phonocardiogram can represent a single cardiac cycle. In at least one embodiment, the clinician knows approximately where each physiological event occurs because the display is “normalized” to a complete cardiac cycle.

The composite phonocardiograms can be time-synchronized averages of the “power” of the acoustic signal within a prescribed frequency bandwidth (e.g., 30-700 Hz). In at least one embodiment, power of the acoustic signal is the root-mean-squared of the acoustic signal. In at least one embodiment, the power can be related to the rectified and filtered acoustic signal which can avoid cancellation artifacts caused by negative oscillation phases of a sampled waveform cancelling positive oscillation phases of another waveform).

As an example, composite phonocardiogram 602 will be discussed in detail. The composite phonocardiogram 602 can have an associated acoustic signal 622 and electrical signal 118.

The electrical signal 118 may include R-wave 618 and T-wave 612. R-wave 618 may indicate depolarization of the left and right ventricles and be used as a trigger to synchronize acoustic signals from the same cardiac cycle. In other words, each R-wave may be used to establish timing of the acoustic signal 622 and where each heart sound (e.g., normal S1 and S2 heart sounds) should occur within each cardiac cycle.

While early electrical features in the electrical signal 620 (ECG waveform) are used to synchronize the sound averaging, other features (e.g., acoustic feature 616 corresponding to an S2 event) in the acoustic signal 622 (PCG waveform) can be added to provide additional temporal fidelity by incorporating later ECG trigger features (e.g., The T-wave 612 can become a secondary trigger point). By using multiple triggering features in the electrical signal 620, synchronization of the heart acoustic features can be improved.

T-wave 612 represents the repolarization of the heart and may be more closely associated with the later heart sounds as the pulmonary and aortic valves close. One could use R-wave 618 as the primary triggering feature (e.g., trigger point 610) and provide further augmentation by adding additional triggering features, such as T-wave 612. In addition, the two different triggering features can be used in a synergistic or complimentary fashion as triggering features.

In one embodiment, alternate triggering features in the ECG waveform can be used in patients with abnormal ECG patterns (e.g., diminished or inverted R-wave). For example, the system may be programmed to recognize an abnormal cardiac cycle which may have a weak R-wave that does not meet the triggering threshold. In such a situation, the system may automatically switch to an alternative triggering feature, such as a T-wave. This is particularly useful because for different cardiac diseases, the primary trigger feature may not be evident. Thus, the ability to automatically switch to a different triggering feature can be advantageous in diagnosing an abnormality.

In at least one embodiment, the time start 608 of the cardiac cycle is sooner than the trigger point 610. The time start 608 can also be set to any electrical feature such as the P-wave 624 or to a predetermined time before the electrical feature.

In at least one embodiment, the acoustic signal 622 can include the acoustic feature 614 (corresponding to an S1), and an acoustic feature 616 (corresponding to an S2). In at least one embodiment, the cardiac cycle includes no greater than four acoustic features. In some examples, the composite phonocardiogram may also reinforce subliminal sounds (e.g., sounds that may not be audible or discernible to the human ear due to low intensity and/or interfering noise). Example subliminal sounds may include S3 heart sounds, S4 heart sounds, or other abnormal sounds from the patient. Electronic stethoscope may present the composite phonocardiogram visually as a waveform that includes the reinforced (e.g., amplified or non-amplified) subliminal sound envelope. Electronic stethoscope may be configured to play back the audible composite phonocardiogram to the user with the previous subliminal or inaudible signal that is enhanced by reinforcement, noise reduction, and/or amplification. In at least one embodiment, the composite phonocardiogram can be continuously displayed and a synthesized sound representing the composite phonocardiogram can be synchronously played through the audio headset.

FIG. 7 illustrates a system 700 in one embodiment. The system 700 comprises electronic stethoscopes 708 communicatively coupled via a wide area network 702 to a server system 704 via an optional proxy server 706. The network topology of the system 700 is a partially connected mesh network. The electronic stethoscopes 708 can be organized into groups of partially connected meshes, and communicate within a mesh group without interacting with the server system 704 or proxy server 706. This type of network topology may be found in environments in which the electronic stethoscopes 708 are spread apart and battery powered, so that they can communicate using relatively short-range wireless communications (e.g., near-field communications). In such environments, a particular one of the electronic stethoscopes 708 may be within communication range of a nearest neighbor. In at least one embodiment, the electronic stethoscopes 708 may transmit detected and/or generated information to a computing device (e.g., a notebook computer, tablet computer, mobile phone, server system 704, or any other computing device) via direct wireless communication or over a wide area network 702.

One or more of the electronic stethoscopes 708 acts as a gateway device 710 providing a communication channel to the server system 704. The electronic stethoscopes 708 that are not the gateway device 710 communicate directly with the gateway device 710, or via the proxy server 706, which communicates on their behalf and on its own behalf with the server system 704. The optional proxy server 706 may improve the performance of the system 700 by mirroring some or all of the state of the server system 704 and thus enabling the electronic stethoscopes 708 to communicate without creating bandwidth or incurring the latency of the wide area network 702. The optional proxy server 706 is typically collocated at a facility or nearby facility to where the electronic stethoscopes 708 are located.

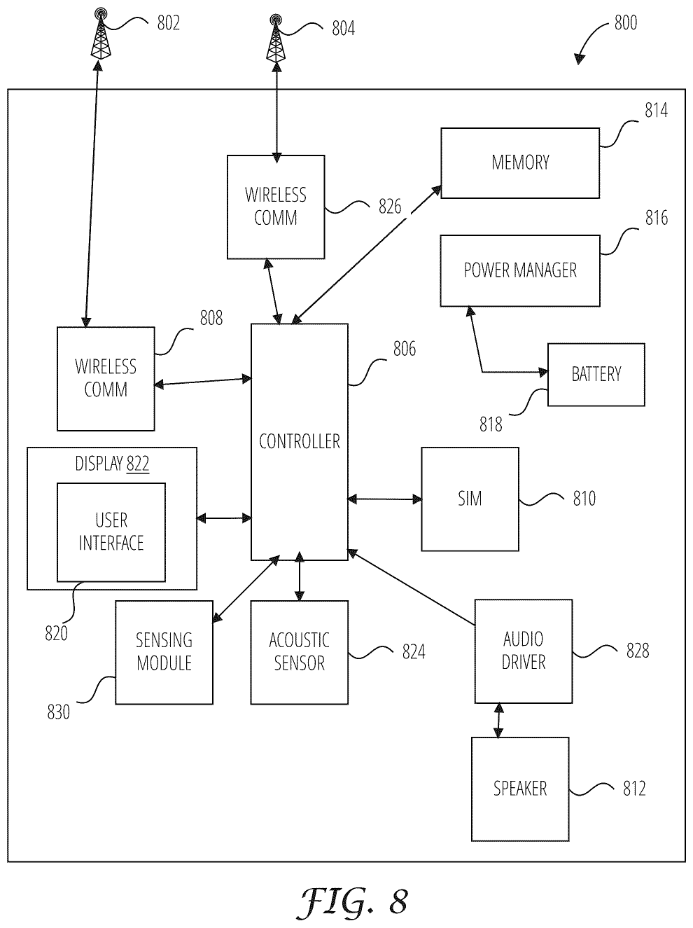

Referring to the electronic stethoscope 800 of FIG. 8, signal processing and system control 806 controls and coordinates the operation of other components as well as providing signal processing for the electronic stethoscope 800. For example, signal processing and system control 806 may extract baseband signals from radio frequency signals received from the wireless communication 808 logic, and processes baseband signals up to radio frequency signals for communications transmitted to the wireless communication 808 logic. Signal processing and system control 806 may comprise a central processing unit, digital signal processor, and/or one or more controllers or combinations of these components.

The wireless communication 808 may further comprise memory 814 which may be utilized by the signal processing and system control 806 to read and write instructions (commands) and data (operands for the instructions).

A human user or operator of the electronic stethoscope 800 may utilize the user interface 820 to receive information from and input information to the electronic stethoscope 800. Images, video and other display information, for example, user interface optical patterns, may be output to the user interface 820, which may be projected onto a display 822 (for example, a liquid crystal display, LED, or e-ink or may utilize other optical output technology).

The user interface 820 may also operate as a user input device, being touch sensitive where contact or close contact by a use's finger or other device handled by the user may be detected by transducers. An area of contact or proximity to the user interface 820 may also be detected by transducers and this information may be supplied to the signal processing and system control 806 to affect the internal operation of the electronic stethoscope 800 and to influence control and operation of its various components.

The acoustic sensor 824 may detect acoustic signals from a heart of the patient as described herein.

Audio signals may be provided to user interface 820 from which signals output to one and more speakers to create pressure waves in the external environment representing the audio. The electronic stethoscope 800 may convert audio phenomenon from the environment into internal electro or optical signals by operating a microphone and audio circuit (not illustrated). User interface 820 may be configured to receive input from a user (e.g., tactile, audio, or video feedback). User interface 820 may include a touch-sensitive and/or a presence-sensitive screen, a voice responsive system, or any other type of device for detecting a command from a user. User interface 820 may include a display 822 for presenting visual information (e.g., a composite phonocardiogram) or audio information (e.g., playback of acoustic information or a composite phonocardiogram). For example, user interface 820 may include a sound generation device configured to present the composite phonocardiogram audibly to a user. The sound generation device may be an electro-acoustic transducer for converting electric signals into sounds (e.g., a speaker 812 or headphone).

In some examples, playback of the acoustic information and/or composite phonocardiogram may be augmented to aid the user in identification of heart sounds. For example, user interface 820 may present a slowed down real-time playback of acoustic information. The slowed playback may incorporate any filtering or analysis described herein. This slowed down audio playback may be useful for young patients or other patients with higher heart rates. This slowed down playback may be synchronized with the actual (i.e., real-time) heart rate such that one slowed down heartbeat occurs within 2, 3, 4, etc. real-time heart beats. Non-synchronized playback may also be provided to the user. The playback speed may be selected by the user via one or more inputs provided by user interface 820.

The electronic stethoscope 800 may operate on power received from a battery 818. The battery 818 capability and energy supply may be managed by a power manager 816.

The electronic stethoscope 800 may transmit wireless signals of various types and range (e.g., cellular, WiFi, Bluetooth, and near field communication (NFC)). The electronic stethoscope 800 may also receive these types of wireless signals. Cellular wireless signals are transmitted and received using wireless communication 808 logic coupled to one or more antenna 802. Shorter-range wireless signals may be transmitted and received via antenna 804 and wireless communication logic 826. Other forms of electromagnetic radiation may be used to interact with proximate devices, such as infrared (not illustrated).

The device may utilize a sensing module 830 which may detect electrical signals (e.g., ECG signals) from two or more electrodes coupled therewith.

A subscriber identity module (SIM 810) may be present in some mobile devices, especially those operated on the Global System for Mobile Communication (GSM) network. The SIM 810 stores, in machine-readable memory, personal information of a mobile service subscriber, such as the subscriber's cell phone number, address book, text messages, and other personal data. A user of the electronic stethoscope 800 can move the SIM 810 to a different and maintain access to their personal information. A SIM 810 typically has a unique number which identifies the subscriber to the wireless network service provider.

The electronic stethoscope 800 may include an audio driver 828 including an audio encoder/decoder for encoding and decoding digital audio files or audio files stored by memory 814, SIM 810, or received in real time via one of the antenna 802, or antenna 804. The audio driver 828 is controlled by the signal processing and system control 806 and decoded audio is provided to one and more speaker 812 to create pressure waves in the external environment representing the audio.

“6LoWPAN” refers to a communication protocol that compresses IPv6 packages for communication by small, low power-devices.

“802.11” refers to a family of wireless communication protocols and technologies commonly referred to as WiFi. Examples of 802.11 are variations such as 802.11a, 802.11b, 802.11g, 802.11ah, and 802.11i.

“Access point” refers to a node that allows users or devices to authenticate to and utilize a network. Access points often implement 802.11 wireless communication.

“Acoustic feature” refers to features in the acoustic signal describing a physiological event in the cardiac cycle.

“Acoustic reading” refers to a single physiological measurement of a sound at a moment of time.

“Acoustic signal” refers to a physiological sound produced by the patient's heart.

“Beacon” refers to wireless devices that communicate location signals indoors, typically without the need for GPS.

“Bluetooth” refers to a family of wireless communication technologies for exchanging data over short distances (using short-wavelength UHF radio waves in the ISM band from 2.4 to 2.485 GHz[3]) from fixed and mobile devices. Variations of Bluetooth are many, including Bluetooth Low Energy, Class 1 Bluetooth (for communications over 100 m, up to 1 km) and Class 2 Bluetooth (10-20m range).

“Bluetooth Low Energy (BLE)” refers to a version of Bluetooth technology that consumes lower power than conventional Bluetooth. BLE is designed for use by portable devices and networking implementations such as Bluetooth Mesh, a Bluetooth topology that allows devices to be connected together, sending/repeating commands from the hub to any connected device. Apple's iBeacon is an example of a BLE application.

“Cardiac cycle” refers to a performance of the human heart from the ending of one heartbeat to the beginning of the next.

“Clinician” refers to a medical professional that has direct contact with a patient. The clinician is a user of the electronic stethoscope and uses the electronic stethoscope to receive biosignals from the patient.

“Composite phonocardiogram” refers to a synchronized view of the phonocardiogram and the electrocardiogram.

“Displayable area” refers to an area that is capable of displaying or activating a pixel. The displayable area does not include areas that are not capable of displaying an image or information.

“Electrical feature” refers to features in the electrical signal describing a physiological event in the cardiac cycle.

“Electrical reading” refers to a single physiological measurement of an electrical signal at a moment of time.

“Electrical signal” refers to a physiological reading of electrical activity of the patient's heart.

“Gateway” refers to a device that operates to bridge communication between two network systems.

“IBeacon” refers to a technology introduced by Apple that uses sensors to locate iOS or Android devices indoors and can send them notifications via Bluetooth Low Energy (BLE).

“IGMP” refers to Internet Group Management Protocol, a communication protocol based on the IP protocol and is used to support group communication. IGMP allows for IP-multicasting that enables the transmission of IP packages to many receivers with one transmission.

“IIOT” refers to Industrial Internet of Things, encompassing connected large-scale machinery and industrial systems such as factory-floor monitoring, HVAC, smart lighting, and security. For example, equipment can send real-time information to an application so operators can better understand how efficiently that equipment is running. Also referred to as Industry 4.0, Industrie 4.0, and Industrial IoT.

“IPSEC” refers to Internet Protocol Security, a set of protocols that provide authentication and encryption to Internet Protocol (IP) packets, adding an extra layer of security on IP communications.

“IPv6” refers to a newer Internet protocol that provides more addresses than the IPv4 protocol. An IPv6 address is a 128-bit alphanumeric string that identifies an endpoint device in the Internet Protocol Version 6 (IPv6) addressing scheme.

“L2TP” refers to Layer 2 Tunneling Protocol, a tunneling protocol used to support virtual private networks (VPNs) or as part of the delivery of services by Internet Service Providers. It does not provide any encryption or confidentiality by itself, relying on an encryption protocol that it passes within the tunnel to provide privacy.

“LPLN” refers to Low Power Lossy Networks, networks comprised of embedded devices with limited power, memory, and processing resources. LPLNs are typically optimized for energy efficiency, may use BLE and can be applied to industrial monitoring, building automation, connected homes, healthcare, environmental monitoring, urban sensor networks, asset tracking, and more.

“MAC layer” refers to media access control sublayer, a layer 2 communication technology that along with the logical link control (LLC) sublayer together make up the data link layer. Within that data link layer, the LLC provides flow control and multiplexing for the logical link while the MAC provides flow control and multiplexing for the transmission medium. These two sublayers together correspond to layer 2 of the OSI model. In devices implementing IEEE 802 standards, the MAC provides a control abstraction of the physical layer such that the complexities of physical link control are invisible to the LLC and upper layers of the network stack. Thus, any LLC block (and higher layers) may be used with any MAC. In turn, the MAC is formally connected to the PHY via a media-independent interface. The MAC is typically integrated with the PHY within the same device package, although in theory any MAC may be used with any PHY, independent of the transmission medium.

“NFC” refers to near field communications, a set of communication protocols that enable two electronic devices, one of which is usually a portable device such as a smartphone, to establish communication by bringing them within 4 cm (1.6 in) of each other. NFC devices are often used in contactless payment systems, similar to those used in credit cards and electronic ticket smartcards and allow mobile payment to replace/supplement these systems. This is sometimes referred to as NFC/CTLS (Contactless) or CTLS NFC. NFC is used for social networking, for sharing contacts, photos, videos or files. NFC-enabled devices can act as electronic identity documents and keycards. NFC offers a low-speed connection with simple setup that can be used to bootstrap more capable wireless connections

“Patient” refers to a human that is being analyzed for heart sounds in a clinical or semi-clinical setting.

“PHY” refers to the physical layer of the OSI model, the circuitry required to implement physical layer functions.

“Real-time” refers to a system in which input data is processed within 100 milliseconds so that it is available virtually immediately as feedback.

“RFID” refers to radio frequency ID, devices and systems that utilize electromagnetic fields to automatically identify and track tags attached to objects. The tags contain electronically stored information. Passive tags collect energy from a nearby RFID reader's interrogating radio waves. Active tags have a local power source (such as a battery) and may operate hundreds of meters from the RFID reader. Unlike a barcode, the tag need not be within the line of sight of the reader, so it may be embedded in the tracked object.

“Sigfox” refers to a low-bandwidth, wireless protocol that provides improved range and obstacle penetration for short messages over some other IoT communication technologies.

“Thread protocol” refers to an IPv6-based, low-power mesh networking technology for IoT products, based on 6LoWPAN.

“Zigbee” refers to short range wireless networking protocol that primarily operates on the 2.4 GHz frequency spectrum. Zigbee devices connect in a mesh topology, forwarding messages from controlling nodes to slaves, which repeat commands to other connected nodes.

### List of Illustrative Embodiments:

1. A method of displaying a portion of an acoustic signal on an electronic stethoscope, comprising:
receiving the acoustic signal from an acoustic sensor of the electronic stethoscope and an electrical signal from a sensing module of the electronic stethoscope;
identifying a trigger point associated with the electrical signal, the electrical signal is derived from a cardiac cycle from a plurality of cardiac cycles, the cardiac cycle is associated with a patient;
generating acoustic information from the acoustic signal;
synchronizing the acoustic information to the trigger point for the cardiac cycle;
generating a phonocardiogram as a composite phonocardiogram based on the synchronized acoustic information, wherein the composite phonocardiogram is generated for the cardiac cycle; and
displaying a representative portion of the composite phonocardiogram on a display of the electronic stethoscope in real-time for a duration of a first period of time, wherein the display is integral to a chest piece of the electronic stethoscope and the chest piece is configured for handheld manipulation by a clinician.
   2. The method of embodiment 1, wherein the display has a displayable area of less than 1000 square millimeters.
   3. The method of any of embodiments 1 to 2, wherein the representative portion corresponds to a single cardiac cycle or an average cardiac cycle of a plurality of cardiac cycles.
   4. The method of embodiment 3, wherein no other cardiac cycles besides the first cardiac cycle are displayed.
   5. The method of embodiment 3 or 4, further comprising:
after the first period of time has elapsed, identifying a second cardiac cycle of the patient;
displaying the second cardiac cycle for a duration equivalent to the first period of time.
   6. The method of any of embodiments 1 to 5, wherein receiving the acoustic signal and the electrical signal comprises recording the acoustic signal and the electrical signal into memory.
   7. The method of embodiment 6, wherein the synchronizing the acoustic information comprises:
resetting the display with respect to the acoustic signal or electrical signal; and
setting a time start based on the trigger point; and
retrieving the acoustic signal after the time start.
   8. The method of embodiment 7, further comprising displaying a cursor bar on the composite phonocardiogram based on the time start.
   9. The method of embodiment 8, displaying the cursor bar across the composite phonocardiogram as a function of time.
   10. The method of any of embodiments 7 to 9, wherein the displaying the composite phonocardiogram comprises:
reading the acoustic signal from the memory from the time start;
wherein the representative portion is from the time start until an end of the first period of time.
   11. The method of any of embodiments 7 to 10, wherein the first period of time is related to a duration of a previously recorded cardiac cycle.
   12. The method of embodiment 11, wherein the previously recorded cardiac cycle immediately precedes the cardiac cycle.
   13. The method of any of embodiments 3 to 12, further comprising:
receiving an indication of a second mode;
in response to the indication,
   determining an average cardiac cycle for a plurality of previously recorded cardiac cycles;
   displaying the average cardiac cycle for a second period of time.
      14. The method of embodiment 13, wherein the second period of time is no greater than 5 seconds.
      15. The method of embodiment 13, wherein the average cardiac cycle is based on a rolling average of the plurality of previously recorded cardiac cycles.
      16. The method of embodiment 13, wherein the average cardiac cycle is a composite of a plurality of cardiac cycles.
      17. The method of any of embodiments 1 to 16, wherein the cardiac cycle comprises no greater than four acoustic features.
      18. The method of embodiment 17, wherein the cardiac cycle comprises an S1 and S2 acoustic feature.
      19. The method of any of embodiments 1 to 18, wherein the first period of time is related to a pulse rate of the patient.
      20. The method of any of embodiments 1 to 19, wherein identifying the trigger point utilizes an embedded Pan Tompkins detection algorithm.
      21. A non-transitory computer-readable storage medium including instructions that, when processed by the electronic stethoscope, configure the electronic stethoscope to perform the method of any of embodiments 1 to 20.
      22. An electronic stethoscope comprising:
a chest piece comprising:
a display integral with a housing of the chest piece;
a sensing module;
an acoustic sensor;
a processor communicatively coupled to the display, the sensing module, a memory, and the acoustic sensor; and
the memory storing instructions that, when executed by the processor, configure the electronic stethoscope to:
   receive an acoustic signal from the acoustic sensor of an electronic stethoscope and an electrical signal from the sensing module of the electronic stethoscope;
   identify a trigger point associated with the electrical signal, the electrical signal is derived from a cardiac cycle from a plurality of cardiac cycles, the cardiac cycle is associated with a patient;
   generate acoustic information from the acoustic signal; synchronize the acoustic information to the trigger point for the cardiac cycle;
   generate a phonocardiogram as a composite phonocardiogram based on the
   synchronized acoustic information, wherein the composite phonocardiogram is generated for the cardiac cycle; and
   display a representative portion of the composite phonocardiogram on the display in real-time for a duration of a first period of time.
      23. The electronic stethoscope of embodiment 22, wherein the composite phonocardiogram is configured to be read by a clinician while being applied to the patient.
      24. The electronic stethoscope of any of embodiments 22 to 23, wherein the housing is configured to contain the display, the sensing module, the acoustic sensor, and processor and memory.
      25. The electronic stethoscope of any of embodiments 22 to 24, wherein the display has a displayable area of less than 1000 square millimeters.
      26. The electronic stethoscope of any of embodiments 22 to 25, further comprising eartips that are acoustically coupled to the chest piece.
      27. The electronic stethoscope of embodiment 26, wherein the eartips are acoustically coupled to the chest piece via a sound transmission device.
      28. The electronic stethoscope of any of embodiments 22 to 27, wherein the representative portion corresponds to a single cardiac cycle or an average cardiac cycle of a plurality of cardiac cycles.
      29. The electronic stethoscope of embodiment 28, wherein no other cardiac cycles besides the first cardiac cycle are displayed.

The electronic stethoscope of any of embodiments 22 to 29, wherein the instructions further configure the electronic stethoscope to:
after the first period of time has elapsed, identify a second cardiac cycle of the patient;
display the second cardiac cycle for a duration equivalent to the first period of time.
   31. The electronic stethoscope of any of embodiments 22 to 30, wherein receiving the acoustic signal and the electrical signal comprises recording the acoustic signal and the electrical signal into the memory.
   32. The electronic stethoscope of embodiment 31, wherein the synchronizing the acoustic information comprises:
reset the display with respect to the acoustic signal or the electrical signal; and
set a time start based on the trigger point; and
retrieve the acoustic signal after the time start.
   33. The electronic stethoscope of any of embodiments 22 to 32, wherein the instructions further configure the electronic stethoscope to display a cursor bar on the composite phonocardiogram based on the time start.
   34. The electronic stethoscope of any of embodiments 22 to 33, wherein the displaying the composite phonocardiogram comprises:
electrical reading the acoustic signal from the memory from the time start;
wherein the representative portion is from the time start until an end of the first period of time.

The electronic stethoscope of any of embodiments 22 to 34, wherein the first period of time is related to a duration of a previously recorded cardiac cycle.

36. The electronic stethoscope of embodiment 35, wherein the previously recorded cardiac cycle immediately precedes the cardiac cycle.
37. The electronic stethoscope of any of embodiments 22 to 36, wherein the instructions further configure the electronic stethoscope to:
receive an indication of a second mode;
in response to the indication,
determine an average cardiac cycle for a plurality of previously recorded cardiac cycles;
display the average cardiac cycle for a second period of time.
   38. The electronic stethoscope of embodiment 37, wherein the average cardiac cycle is based on a rolling average of the plurality of previously recorded cardiac cycles.
   39. The electronic stethoscope of any of embodiments 22 to 38, wherein the first period of time is related to a pulse rate of the patient.
   40. A system comprising:
the electronic stethoscope of any of embodiments 22 to 39;
a server system, wherein the electronic stethoscope is configured to transmit the representative portion or the composite phonocardiogram to the server system.

What is claimed is:

1. A method of displaying a portion of an acoustic signal detected using an electronic stethoscope, comprising:
receiving the acoustic signal from an acoustic sensor of the electronic stethoscope and an electrical signal from a sensing module of the electronic stethoscope;
identifying a trigger point associated with the electrical signal, the electrical signal is derived from a cardiac cycle from a plurality of cardiac cycles, the cardiac cycle is associated with a patient;
generating acoustic information from the acoustic signal;
synchronizing the acoustic information to the trigger point for the cardiac cycle;
generating a phonocardiogram as a composite phonocardiogram based on the synchronized acoustic information, wherein the composite phonocardiogram is generated for the cardiac cycle; and
displaying a representative portion of the composite phonocardiogram on a display in real-time for a duration of a first period of time.

2. The method of claim 1, wherein the display has a displayable area of less than 1000 square millimeters.

3. The method of claim 1, wherein the representative portion corresponds to a single cardiac cycle or an average cardiac cycle of a plurality of cardiac cycles.

4. The method of claim 3, further comprising:
after the first period of time has elapsed, identifying a second cardiac cycle of the patient;
displaying the second cardiac cycle for a duration equivalent to the first period of time.

5. The method of claim 1, wherein receiving the acoustic signal and the electrical signal comprises recording the acoustic signal and the electrical signal into memory, and
wherein the synchronizing the acoustic information comprises:
resetting the display with respect to the acoustic signal or electrical signal; and
setting a time start based on the trigger point; and
retrieving the acoustic signal after the time start.

6. The method of claim 5, further comprising displaying a cursor bar on the composite phonocardiogram based on the time start.

7. The method of claim 6, displaying the cursor bar across the composite phonocardiogram as a function of time.

8. The method of claim 7, wherein the displaying the composite phonocardiogram comprises:
reading the acoustic signal from the memory from the time start;
wherein the representative portion is from the time start until an end of the first period of time.

9. The method of claim 7, wherein the first period of time is related to a duration of a previously recorded cardiac cycle.

10. The method of claim 9, wherein the previously recorded cardiac cycle immediately precedes the cardiac cycle.

11. The method of claim 3, further comprising:
receiving an indication of a second mode;
in response to the indication,
determining an average cardiac cycle for a plurality of previously recorded cardiac cycles;
displaying the average cardiac cycle for a second period of time.

12. The method of claim 11, wherein the average cardiac cycle is based on a rolling average of the plurality of previously recorded cardiac cycles.

13. An electronic stethoscope comprising:
a chest piece comprising:
a display integral with a housing of the chest piece;
a sensing module;
an acoustic sensor;
a processor communicatively coupled to the display, the sensing module, a memory, and the acoustic sensor; and
the memory storing instructions that, when executed by the processor, configure the electronic stethoscope to:
receive an acoustic signal from the acoustic sensor of an electronic stethoscope and an electrical signal from the sensing module of the electronic stethoscope;
identify a trigger point associated with the electrical signal, the electrical signal is derived from a cardiac cycle from a plurality of cardiac cycles, the cardiac cycle is associated with a patient;
generate acoustic information from the acoustic signal;
synchronize the acoustic information to the trigger point for the cardiac cycle;
generate a phonocardiogram as a composite phonocardiogram based on the synchronized acoustic information, wherein the composite phonocardiogram is generated for the cardiac cycle; and
display a representative portion of the composite phonocardiogram in real-time for a duration of a first period of time.

14. The electronic stethoscope of claim 13, wherein the composite phonocardiogram is configured to be read by a clinician while being applied to the patient.

15. The electronic stethoscope of claim 13, wherein the display has a displayable area of less than 1000 square millimeters.

16. The electronic stethoscope of claim 13, further comprising eartips that are acoustically coupled to the chest piece, wherein the eartips are acoustically coupled to the chest piece via a sound transmission device.

17. The electronic stethoscope of claim 13, wherein no other cardiac cycles besides the first cardiac cycle are displayed.

18. The electronic stethoscope of claim 13, wherein the instructions further configure the electronic stethoscope to:
after the first period of time has elapsed, identify a second cardiac cycle of the patient;
display the second cardiac cycle for a duration equivalent to the first period of time.

19. The electronic stethoscope of claim 13, wherein receiving the acoustic signal and the electrical signal comprises recording the acoustic signal and the electrical signal into the memory, and
wherein the synchronizing the acoustic information comprises:
reset the display with respect to the acoustic signal or the electrical signal; and
set a time start based on the trigger point; and
retrieve the acoustic signal after the time start.
